(19) United States
(12) Patent Application Publication
(10) Pub. No.: US 2026/0248976 A1
(43) Pub. Date: Aug. 27, 2026

(54) DEVICE AND METHOD FOR ENSURING STERILITY

(71) Applicant: OPTIMA pharma GmbH, Schwäbisch Hall (DE)

(72) Inventors: Marcel Holl, Schwäbisch Hall (DE); Florian Weippert, Schwäbisch Hall (DE)

(73) Assignee: OPTIMA pharma GmbH, Schwäbisch Hall (DE)

(21) Appl. No.: 19/651,055

(22) Filed: Apr. 17, 2026

Related U.S. Application Data
(63) Continuation of application No. PCT/EP2024/072455, filed on Aug. 8, 2024.

(30) Foreign Application Priority Data
Oct. 17, 2023 (DE) 102023128342.1

(51) Int. Cl. A61L 2/26; A61L 2/07

(52) U.S. Cl. CPC A61L 2/26; A61L 2/07

(57) ABSTRACT

The invention relates to a device and a method for ensuring the sterility of objects, products, object-contacting components, and/or product-contacting components in a cleanroom, wherein in the cleanroom an air flow is guided over a component (20, 30) to an object, a product, object-contacting component and/or a product-contacting component, wherein a cover (10) made of a sterilizable, in particular autoclavable, material can be arranged on the component (20, 30) or upstream of the component (20, 30) in such a way that the cover (10) prevents contamination of the air flow guided over the component (20, 30) by the component (20). The invention further relates to the use of a cover (10) for ensuring sterility in a cleanroom.

### CROSS-REFERENCE TO RELATED APPLICATIONS

This application is continuation of PCT/EP 2024/072455, filed Aug. 8, 2024, and claims priority to German Patent Application No. 102023128342.1, filed Oct. 17, 2023, the entire contents of both of which are hereby incorporated by reference.

### FIELD OF APPLICATION AND PRIOR ART

The invention relates to a device and a method for ensuring the sterility of objects in a cleanroom. The invention further relates to the use of a cover to ensure the sterility of objects in a cleanroom.

In the context of this application, a decontaminated unit that allows its interior to be isolated from the external environment is referred to as an isolator. Isolators are used in particular in the manufacture, processing, and handling of pharmaceutical products, biopharmaceutical products, biological products, toxic products, and/or other highly sensitive products. A cleanroom is provided inside the isolator. Equipment and/or materials that are manufactured, processed, or otherwise handled in the cleanroom-including packaging materials such as containers into which the products are filled, closure elements such as stoppers used to seal the containers, or similar items-are referred to as “objects” in the context of the application.

High standards of sterility are required, particularly for the handling (manufacture, processing, and/or manipulation) of pharmaceutical and/or biopharmaceutical products. The requirements for a cleanroom in the pharmaceutical industry are set forth, among other places, in Annex 1 of the EU GMP Guidelines on the Manufacture of Sterile Medicinal Products (EU Annex 1).

### OBJECT AND SOLUTION

The object of the invention is to provide a device and a method for ensuring the sterility of products and/or objects during their handling in a cleanroom, particularly in a cleanroom used for the manufacture and/or filling of sterile pharmaceuticals.

This problem is solved by the device and method having the features of claims 1 and 9, as well as the use having the features of claim 14. Advantageous embodiments are described in the dependent claims.

According to a first aspect, a device is provided for ensuring the sterility of objects, products, object-contacting components and/or product-contacting components in a cleanroom, wherein in the cleanroom an airflow is guided over a component located toward an object, a product, an object-contacting component and/or a product-contacting component. The device comprises a cover made of a sterilizable material, in particular one that is autoclavable, wherein the cover can be arranged on the component or upstream of the component in such a way that the cover prevents contamination of the airflow guided over the component.

The terms “a,” “an,” etc., are used in the context of the application solely as indefinite articles and not as numerals. In particular, the device may include more than one cover in certain embodiments.

The device makes it possible to arrange components that are only partially sterilizable-for example, components that cannot be autoclaved or can only be autoclaved to a limited extent due to their nature-in an air stream towards objects, products, object-contacting components, and/or product-contacting components, without the risk that the air stream will reach a surface of the components that cannot be autoclaved or can only be autoclaved to a limited extent and be contaminated by it.

These components include, for example, parts of kinematic systems or handling systems, such as lifting shafts, beams, joints, or arms, or other components that are permanently installed in an isolator or cleanroom and/or are placed inside the isolator or cleanroom prior to decontamination.

The cover makes it possible to cover critical areas of the components with a second layer of material—particularly one that is autoclavable—and/or to redirect an airflow so that it does not come into contact with the surface of the components.

During use, the airflow thus comes into contact only with the cover, which can be sterilized beforehand, thereby preventing the risk of contamination or at least reducing it to an acceptable level.

In other words, the cover allows for airflow in a cleanroom to be directed in such a way as to ensure sterility, so that the filtered air supplied to the cleanroom is deflected exclusively by sterilized-specifically, autoclaved-surfaces before it reaches the product, the objects, specifically the surfaces of the objects that come into contact with the product, object-contacting components and/or product-contacting components that come into contact with the product or the objects, such as filling needles or stopper tracks.

The design of the cover can be selected by a qualified professional based on the component to be covered. The covers are preferably designed and/or arranged in such a way that they do not perform a load-bearing function and therefore do not have to withstand any forces. The cover can therefore be designed with thin walls and is lightweight. If necessary—for example, during disassembly—the cover can be removed from the isolator and, after sterilization (particularly steam sterilization in an autoclave), reinserted into the isolator following its decontamination, or replaced with an alternative component.

In one embodiment, the cover is designed to comprise two or more parts and to enclose at least a section of the component. The cover can be adapted to the shape of a component in such a way that it encloses the component airtight in its critical area.

Depending on the circumstances, the cover can be attached to the component by at least one of a form-fit connection, a force-fit connection, a snap-fit connection, a magnetic connection, and/or a plug-in connection. In an embodiment, the cover is attached to the component indirectly, wherein parts of the cover are brought into engagement with one another, and the cover is thus secured to a component located between the parts of the cover. In other embodiments, the cover is attached directly to the component, for example by snapping into place and/or securing it to the component using a magnetic connection. In some embodiments, the cover includes fastening elements for tool-free, one-time attachment of the cover to the component. Examples of fasteners include hooks for a snap-fit connection or magnets for a magnetic connection. A tool-free fastening is a fastening method that allows the cover to be attached to the component without the use of tools. In some embodiments, the cover can be gripped or handled using a tool. In some embodiments, it is not possible to remove the cover from the component without tools.

In some embodiments, the design is such that the cover can be attached to the component using a kinematic system located within the isolator, without the need for manual intervention via a glovebox. This prevents the cover from being contaminated by gloves, which allow manual access to the cleanroom.

Alternatively or additionally, some embodiments provide that the cover includes a handling section—specifically, a handling section projecting from a surface, such as a tab, a handle, or a grip—and/or a visually identifiable marked area for handling the cover. The handling section is designed and/or positioned in such a way that it allows for the manual installation of the cover inside the isolator—either directly, using a tool, and/or using a shielding device—without any contamination of the surfaces located within the airflow.

In an embodiment, the outer contour of the cover matches the outer contour of the component on which the cover is mounted. In other embodiments, the outer contour of the cover is designed to differ from the outer contour of the component. The cover can thus be used specifically to make localized aerodynamic adjustments within the cleanroom without altering the structural mechanics of the associated component, and in particular without changing the component's design.

The cover can be customized to meet the specific requirements of each use application. In some embodiments, the cover is designed to be made of an autoclavable material, specifically metal and/or plastic.

According to a second aspect, a method for ensuring the sterility of objects, products, object-contacting components, and/or product-contacting components in a cleanroom, wherein in the cleanroom an air flow is guided over a component arranged toward an object, a product, object-contacting component and/or a product-contacting component, wherein a cover made of a sterilizable material, in particular autoclavable material, is arranged on the component or upstream of the component in such a way that the cover prevents contamination of the airflow guided over the component by the component.

Depending on the use application, the cover can be mounted manually by a person—for example, using a tool through a glove port—or fully or partially automated using a kinematic system located within the cleanroom.

In an embodiment, the cover is designed comprise two or more parts, and the component is enclosed by the cover, at least in sections. The cover is placed around the component in such a way that it encloses it like a sleeve.

Depending on the use application, the cover is attached to the component by at least one of a form-fit connection, a force-fit connection, a snap-fit connection, a magnetic connection, and/or a push-fit connection. One can choose the connection type that is appropriate for your specific application. In embodiments. the connection design is such that the cover can be repeatedly attached in a non-destructive manner using a fully or partially automated kinematic system. In other embodiments, disassembly is only possible by destroying the cover.

In typical applications, the cover is brought into the cleanroom after the cleanroom has been decontaminated and attached to the component or upstream of the component. In some embodiments, the cover is transferred into the cleanroom through a port provided on the cleanroom. In some embodiments, the cover is designed so that it can be placed inside an autoclavable bag and, using the bag, made available at a port for transfer to the cleanroom.

Using the cover, it is possible to retrofit an existing cleanroom, for example, to better adapt it to increased sterility requirements.

According to a third aspect, the use of a cover made of a sterilizable, particularly autoclavable, material is provided to ensure the sterility of objects, products, object-contacting components and/or product-contacting components, wherein the cover is arranged on or upstream of a component located in the cleanroom in such a way that contamination of an air flow guided from the component to an object, a product, an object-contacting component and/or a product-contacting component is prevented by the cover.

As described above, the use of a cover allows creating of an airflow pattern in a cleanroom to ensure sterility in such a way that the filtered air supplied to the cleanroom is deflected exclusively by sterilized surfaces—particularly those that have been autoclaved—before it reaches the product and product-contacting surfaces of objects.

The cover can be customized by a professional to suit the specific use application.

### BRIEF DESCRIPTION OF THE DRAWINGS

Further advantages and aspects of the invention are apparent from the claims and from the description of exemplary embodiments of the invention, which are explained below with reference to the figures.

FIG. 1 a first exemplary embodiment of a device for ensuring sterility, comprising a cover mounted on a solid arm of a kinematic system;

FIG. 2 the device of FIG. 1 with the arm in an exploded view of the cover;

FIG. 3 the arm as shown in FIG. 1, without the device;

FIG. 4 a second exemplary embodiment of a device for ensuring sterility, comprising a cover mounted on an arm of a closing station;

FIG. 5 the cover from FIG. 4 in an exploded view;

FIG. 6 an alternative exemplary embodiment of a cover shown in cross-section; and

FIG. 7 a cross-sectional view of another exemplary embodiment of a cover.

### DETAILED DESCRIPTION OF THE EXEMPLARY EMBODIMENTS

In the figures, the same reference symbols are used for identical or similar elements.

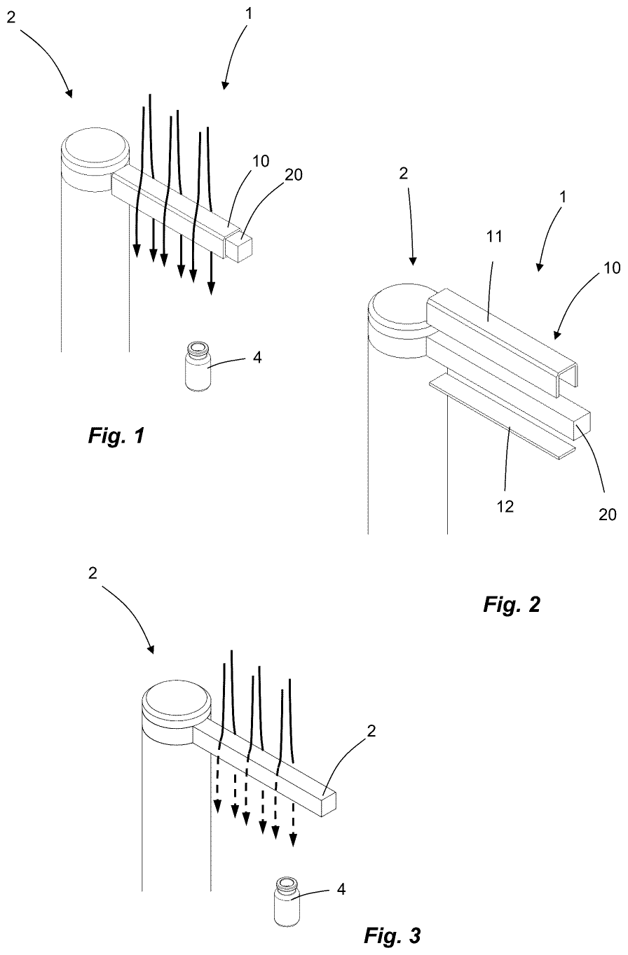

FIGS. 1 and 2 show a first exemplary embodiment of a device 1 for ensuring the sterility of objects and/or products—not shown in the figures—in a cleanroom.

The device 1 comprises a cover 10 made of a sterilizable material, in particular metal or plastic. The cover 10 shown in FIGS. 1 and 2 can be attached to a component 20 in such a way that the cover 10 prevents contamination of an airflow passing over the component 20 by the component 20.

The component 20, as shown in FIGS. 1 and 2, is a solid arm of a kinematic system 2. The cover 10 can be designed in such a way that with the cover 10 the functionality of the kinematic system 2 is not altered, or at most alters it in a manner that is insignificant for the use application.

As shown in FIG. 2, the cover 10 depicted comprises two parts: a first U-shaped part 11 and a second part 12, which can be connected to the first part 11 to form a closed enclosure for the component designed as an arm 20.

FIG. 1 shows, in a highly schematic manner using arrows, primary air supplied from above that is deflected by the arm 20. Due to the cover, the deflected air is not contaminated before it reaches an object shown schematically in FIG. 1 as container 4.

FIG. 3 shows the arm 20 of kinematic system 2 without the cover 10, with primary air supplied from above and deflected by the arm 20 also shown very schematically using arrows. The deflected air may become contaminated due to contact with the arm 20, so that the air directed toward the object shown schematically as container 4 in FIG. 3 can no longer be referred to as primary air.

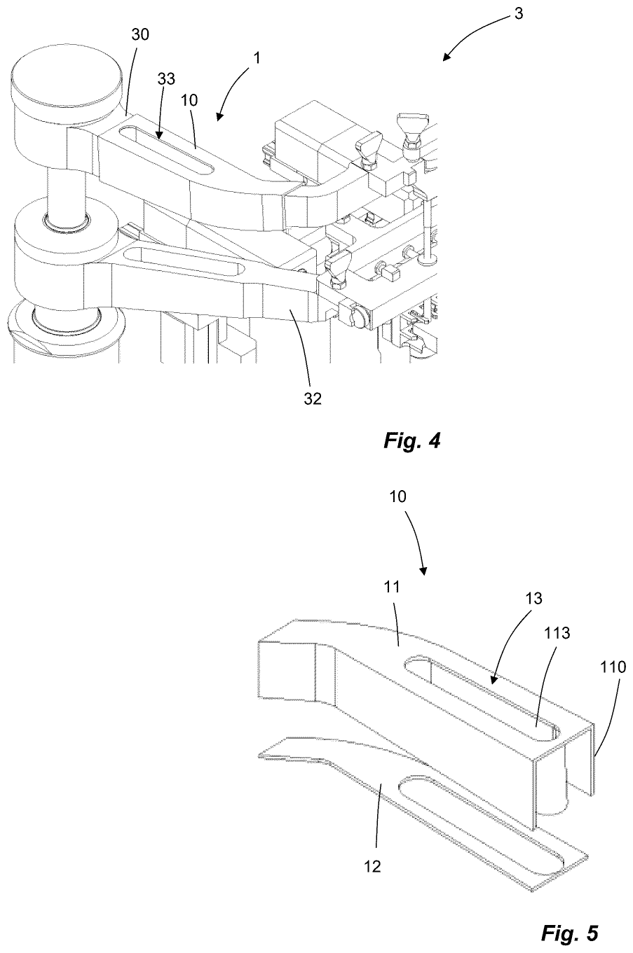

FIGS. 4 and 5 show a second exemplary embodiment of a device 1 for ensuring sterility, comprising a cover 10.

The cover 10 shown in FIGS. 4 and 5 can also be attached to a component 30 in such a way that the cover 10 prevents contamination of an airflow guided over the component 30.

The component 30, as shown in FIGS. 4 and 5, is an arm of a closing station in the form of a stopper inserter 3. The arm 30 has an opening 33 for air passage. In the illustrated exemplary embodiment, the cover 10 for the component 30 is designed in a similar manner and also features an opening 13. The stopper inserter 3 shown here has a second arm 32; in the exemplary embodiment shown, no cover is provided on the second arm 32. In other embodiments, a cover is also provided on the second arm 32.

As can be seen in FIG. 5, the cover 10 shown in FIGS. 4 and 5 is also a two-piece assembly, comprising a first part 11 and a second part 12, which can be connected to the first part 11, thereby providing a closed enclosure for the arm-shaped component 30, including a wall that defines the opening 33. For this purpose, the first part 11 shown in the illustrated exemplary embodiment has a substantially U-shaped outer wall 110 and an inner wall 113 shaped to correspond to the wall bounding the opening 33.

In the exemplary embodiments shown in FIGS. 1, 2, 4, and 5, the outer contours of the covers 10 correspond, at least substantially, to the outer contours of the components 20, 30 on which the covers 10 are respectively mounted.

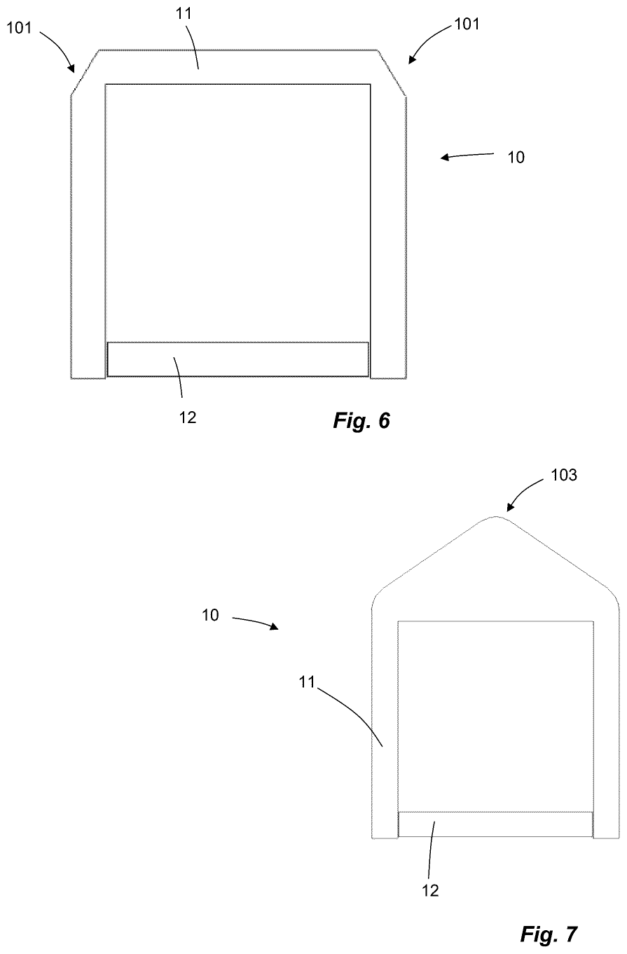

FIGS. 6 and 7 each show, in a cross-sectional view, an embodiment of a cover 10 similar to the cover 10 shown in FIGS. 1 and 2. Like the cover shown in FIG. 1, the covers 10 shown in FIGS. 6 and 7 each comprise a first, U-shaped part 11 and a second part 12 that can be connected thereto, which together form a closed shell.

Unlike the design shown in FIGS. 1 and 2, in the design shown in FIG. 6, the corners 101 of the first part 11 are chamfered.

In the configuration shown in FIG. 7, a projection 102 is provided on an upward-facing side of the first part 11.

The designs shown schematically in FIGS. 6 and 7 make it possible to make local aerodynamic adjustments within the cleanroom by altering the outer contour of the covers 10, without modifying the corresponding component to which the covers 10 are attached.

The designs shown are merely examples, and numerous variations are possible.

What is claimed is:

1. A device for ensuring the sterility of objects, products, object-contacting components, and/or product-contacting components in a cleanroom, wherein in the cleanroom an air flow is guided over a component toward an object, a product, an object-contacting component and/or a product-contacting component, the device comprising a cover made of a sterilizable material, wherein the cover can be arranged on the component or upstream of the component in such a way that the cover prevents contamination of the air flow guided over the component by the component.

2. The device according to claim 1, wherein the cover comprises two or more parts and encloses at least a section of the component.

3. The device according to claim 1, wherein the cover can be secured to the component by at least one of a form-fit connection, a force-fit connection, a snap-fit connection, a magnetic connection, and a plug-in connection.

4. The device according to claim 3, wherein the cover comprises fastening elements for tool-free, one-time fastening of the cover to the component.

5. The device according to claim 1, wherein the cover comprises a handling section.

6. The device according to claim 5, wherein the handling section is a handling section projecting from a surface, such as a tab, a handle, or a grip, and/or the handling section is a visually identifiable marked area.

7. The device according to claim 1, wherein an outer contour of the cover differs from an outer contour of the component.

8. The device according to claim 1, wherein the cover made is made of an autoclavable, material.

9. A method for ensuring the sterility of objects, products, object-contacting components and/or product-contacting components in a cleanroom, wherein in the cleanroom an air flow is guided cleanroom over a component located in the cleanroom toward an object, a product, an object-contacting component and/or a product-contacting, wherein a cover made of a sterilizable material is arranged on the component or upstream of the component in such a way that the cover prevents contamination of the air flow guided over the component by the component.

10. The method according to claim 9, wherein the cover comprises two or more parts and the component is enclosed by the cover at least in sections.

11. The method according to claim 9, wherein the cover is secured to the component by use of at least one of a form-fit connection, a force-fit connection, a snap-fit connection, a magnetic connection, and a plug-in connection.

12. The method according to claim 9, wherein, after decontamination of the cleanroom, the cover is brought into the cleanroom and secured to the component or upstream of the component.

13. The method according to claim 9, wherein the cover made is made of an autoclavable, material.

14. Use of a cover made of a sterilizable material for ensuring the sterility of objects, products, object-contacting components, and/or product-contacting components in a cleanroom, wherein in the cleanroom an air flow is guided over a component to an object, a product, object-contacting component and/or a product-contacting-component, wherein the cover is arranged on the component or upstream of the component in such a way that the cover prevents contamination of the air flow guided over the component by the component.

15. The use according to claim 14, wherein the cover made is made of an autoclavable material.
